(19) United States
(12) Patent Application Publication
(10) Pub. No.: US 2024/0226056 A1
(43) Pub. Date: Jul. 11, 2024

(54) Sleep or Post-Sleep Performance

(71) Applicant: Vanda Pharmaceuticals Inc., Washington, DC (US)

(72) Inventors: Mihael H Polymeropoulos, Potomac, MD (US); Christos Polymeropoulos, Potomac, MD (US); Changfu Xiao, Washington, DC (US)

(21) Appl. No.: 18/613,524

(22) Filed: Mar. 22, 2024

Related U.S. Application Data
(63) Continuation of application No. 17/272,569, filed on Mar. 1, 2021.
(60) Provisional application No. 62/730,467, filed on Sep. 12, 2018.

(51) Int. Cl. A61K 31/343; A61P 25/20

(52) U.S. Cl. CPC A61K 31/343; A61P 25/20

(57) ABSTRACT

The invention relates generally to improving sleep, post-sleep performance, or both and, more particularly, to so improving without inducing post-sleep effects that would impair an individual's ability to operate machinery or a motor vehicle.

### CROSS-REFERENCE TO RELATED APPLICATIONS

This application is a continuation application of co-pending U.S. patent application Ser. No. 17/272,569, filed 1 Mar. 2021, which is the US National Phase of International Application Ser. No. PCT/US19/50785, filed 12 Sep. 2019, which claims the benefit of then-co-pending US Provisional Patent Application Ser. No. 62/730,467, filed 12 Sep. 2018, each of which is hereby incorporated herein as though fully set forth.

### BACKGROUND

Improving one or both of the quality and duration of sleep may be accomplished through the use of medicines that have been established as being safe and effective for doings so. Many such medicines act directly by reducing wakefulness, i.e., inducing a soporific effect. This soporific effect may, in whole or in part, account for the effectiveness of such medicines.

The soporific effect carries with it a potential liability to the extent is persists beyond the normal sleep period and is manifest during the course of the next-day activities of the individual being treated. In such situations, these effects during the day following treatment create the potential to adversely affect an individual's ability to perform various tasks that require wakefulness to be performed in a safe manner, i.e., without creating potential harm to self or others as a result of reduced alertness and residual sleepiness. Notably, many such medicines, although effective to improve sleep quality or duration, are known to affect an individual's ability to safely operate machinery or an automobile. Specifically, the marketing of many such medicines is limited though explicit warnings as to their use when the individual may have to engage in such activities, i.e., must avoid the possibility for driving impairment or operate machinery.

For example, AMBIEN® prescribing information includes a warning that “higher morning blood levels following use of the 10 mg dose increase the risk of next day impairment of driving and other activities that require full alertness.” These effects are worsened with the co-administration of ZOLPIDEM™ or other central nervous system depressants, with the prescribing information warning that such concomitant use “may increase drowsiness and psychomotor impairment, including impaired driving ability:” Similarly, the prescribing information for BELSOMRA® warns of a “[r]isk of impaired alertness and motor coordination, including impaired driving,” with such risk increasing with increased dose. Individuals taking a 20 mg dose of

BELSOMRA® are explicitly cautioned against “next-day driving and other activities requiring complete mental alertness.” Clinical studies have shown “clinically meaningful impaired driving performance in some subjects,” resulting in a warning that even patients taking lower doses of BELSOMRA® should be cautioned about the potential for impaired driving, due to variability in individual sensitivity.

### SUMMARY

The present invention relates to the preferential use of tasimelteon relative to other medicines known to be useful for treating sleep disorders, particularly those capable of inducing next-day soporific effects, in patients that intend to or may be operating motor vehicles or machinery in a post-sleep (e.g., next-day) period following treatment with the sleep aid. As discussed herein, the discovery of the preferential use of tasimelteon in such circumstances arises from the unexpected results from clinical studies designed to assess next-day liability from tasimelteon use.

The present invention, thus, includes a method of improving sleep, post-sleep performance, or both, in an individual being assessed for treatment with, or being treated with, a medicine effective to achieve such an improvement comprising first determining whether the individual intends to operate a motor vehicle or machinery during the post-sleep period following treatment. Once this determination is made and it is determined that individual intends to operate a motor vehicle or machinery during the post-sleep period following treatment, the individual is then treated with tasimelteon by orally administering an amount thereof effective to improve sleep, post-sleep performance, or both. Improving sleep may include improving one or both of sleep quality and sleep duration.

On the other hand, if no such determination is made, the individual is then treated with any medicine known to improve sleep (e.g., sleep quality or sleep duration) and/or post-sleep performance by administering an amount thereof effective to achieve one or both of such outcomes. Thus, this aspect of the invention can include determining that that the individual intends to or may operate a motor vehicle or machinery during the period following treatment and, accordingly, treating the individual by administration of 20 mg of tasimelteon before bedtime.

As a result, the present invention can encompass, in a method consisting of treating an individual who needs to avoid driving impairment or to operate machinery during the day following, administration of a medicine to improve sleep and/or post-sleep performance, the improvement comprising the use of 20 mg of tasimelteon administered before bedtime as said medicine.

Medicines known to improve sleep (e.g., one or both of sleep quality and sleep duration) include, for example, benzodiazepines (e.g., diazepam, estazolam, ctizolam, flurazepam, lorazepam, midazolam, nitrazepam, nitrazolam, quazepam, temazepam, and triazolam), barbituates (e.g., amobarbital, pentobarbital, phenobarbital, secobarbital, and sodium thiopental), melatonin, melatonin agonists (c.g., agomelatine, piromelatine, ramelteon, and tasimelteon), and non-benzodiazepine “z-drugs” (e.g., zolpidem, zopiclone, eszopiclone, and zaleplon). Similarly; the amounts of these agents and the regimens for administering these agents to improve sleep are well known, e.g., through the prescribing information that the marketers of these medicines have made publicly available.

Similarly known are the diagnostic criteria for selecting patients for treatment with sleep aids, including medicines impacting sleep quality and sleep duration. As noted above, the nature of the safety warnings that accompany the prescribing information for many sleep aids require a health care professional who is diagnosing a patient and selecting a medicine for use in treatment understand the nature of the patient's activities that might be impacted were the patient to be prescribed a medicine that might have next-day soporific effects following use the preceding night.

Hence, in order for a healthcare professional (e.g., attending physician) to properly inform a patient of any post-sleep liability, it may be necessary for the attending healthcare professional for the healthcare professional to determine whether the patient will be operating a motor vehicle, for which a prescribed medicine might impair the ability to drive or operate machinery. As used herein, machinery references the types of mechanical or electro-mechanical devices or contrivances for which mental alertness in their operation by an individual may determine whether the use of the machinery can be undertaken without increased risk of harm to the operating individual or to others.

To assure the safety of an individual being considered for treatment with a medicine to address a sleep disorder, it may be necessary to caution the individual against next- day driving or machinery operation or to limit access to potentially more effective medicines arising from the safety concerns based upon next-day effects. The present invention, therefore, affords an alternative to the current treatment practices in which tasimelteon may be selected for administration once a determination is made that an alternative therapy would present post-sleep (e.g., next-day) liabilities for an individual being treated who may be operating a motor vehicle or machinery:

As noted above, one aspect of the present invention can involve the use of tasimelteon, also referred to as HETLIOZ®. Pharmaceutical compositions containing tasimelteon and uses of tasimelteon have been described in the art. Tasimelteon is approved for use as a human medicine for the treatment of Non-24-Hour Sleep-Wake Disorder (Non-24) and is available in a 20 mg unit pharmaceutical dosage form (capsules), indicated for use prior to bedtime at the same time every night. Pharmacologically; tasimelteon is an agonist of the MTIR and MT2R melatonin receptors in the suprachiasmatic nucleus (SCN), the region of the brain associated with the biological clock. Engagement of these receptors by melatonin is believed to regulate circadian rhythms, including the sleep/wake cycle. Consistent with its receptor binding profile, tasimelteon demonstrates potent chronobiotic activity in preclinical models of acute phase-shifting and chronic re-entrainment.

Tasimelteon per se is claimed in U.S. Pat. No. 5,856,529 in claim 7 thereof. The '529 patent contains further claims, including claims to a genus of compounds of which tasimelteon is a member, as well as claims to the use of this genus in treating sleep disorders, as well as circadian rhythm disorders, by administering an effective amount of tasimelteon. The patent describes tasimelteon as a melatonin agonist and further speculates that melatonin agonists would be useful for the further study of melatonin receptor interactions as well as in the treatment of conditions affected by melatonin activity: The patent lists depression, jet lag, work-shift syndrome, and sleep disorders, among other possible therapeutic uses. Elsewhere, the patent discloses that compounds within genus of compounds of which tasimelteon is a member are useful as melatonergic agents in the treatment of sleep disorders, seasonal depression, shifts in circadian cycles, melancholia, stress, appetite regulation, benign prostatic hyperplasia, and related conditions.

In addition to tasimelteon's approved dosing of 20 mg per day prior to bedtime at the same time every day; U.S. Patent Application Publication No. 20090105333 reports the discovery that effective human doses for tasimelteon can range from 10 to 100 mg/day for contemplated uses in sleep disorders and circadian rhythm disorders, with a further description that the exact dosing may be dependent upon particle size of the tasimelteon and the body size of the patient being treated. The patent describes also describes a 20 mg oral unit dosage form for tasimelteon and a clinical trial using tasimelteon in 10 mg. 20 mg, 50 mg, and 100 mg daily doses, from a tasimelteon study of subjects with a 5-hour advance in their sleep-wake cycle, i.e., the type of sleep-wake cycle advance that might be experienced by a subject traveling by jet aircraft across the Atlantic Ocean from New York to London. The results of this study indicate that treatment relative to placebo produces positive outcomes for shifting dim light melatonin onset and sleep efficacy.

### DETAILED DESCRIPTION

The effectiveness of tasimelteon in addressing post-sleep (e.g., next-day) effects found during the use of other medicines prescribed as sleep aids can be demonstrated clinically. Specifically, a clinical trial to study the effects of tasimelteon on driving finds that a 20 mg dose of tasimelteon does not affect next-day driving and does not induce results significantly different from a negative placebo control. The same study finds that zopiclone, when employed as a positive control, does significantly impair driving ability; as compared to the negative placebo control.

In this study, 48 healthy volunteers operate an automobile driving simulator the moming after being administered a bedtime dose of tasimelteon 20 mg, zopiclone 7.5 mg, or placebo. Volunteers are instructed to operate the driving simulator for about one hour with a speed of 55 mph while maintaining lane position.

Table 1 below shows the timing of relevant steps in the study design with respect to both clock time and relative to the administration of tasimelteon, zopiclone, or placebo.

TABLE 1

| procedure | clock time¹ | hours post-dose |
|---|---|---|
| dosing | 23:00 | 0 |
| bedtime/awakening | 23:20/07:00 | 0.5/8 |
| CRCDS² | 08:00 | 9 |

¹Actual times may vary based on dosing time, which is 30 minutes prior to the subject's target bedtime.
²Cognitive Research Corporation Driving Stimulator.

Driving performance is assessed by a number of validated measures, including standard deviation from lateral position (SDLP), a measure of lane weaving. The results are shown in Table 2 below.

TABLE 2

| | SDLP | | |
|---|---|---|---|
| | Difference³ | | |
| Treatment Comparison | (cm) | 95% CI | p-value |
| Tasimelteon vs Placebo | 1.22 | (−0.29, 2.74) | p = 0.1119 |
| Zopiclone vs Placebo | 4.14 | (2.60, 5.68) | p < 0.0001 |
| Tasimelteon vs | −2.92 | (−4.43, −1.41) | p = 0.0002 |
| Zopiclone | | | |

³Least squares means.

As can be seen, tasimelteon 20 mg demonstrates no post-sleep (next-day) driving impairment compared to placebo when evaluated nine hours after dosing, while zopiclone 7.5 mg is associated with a meaningful and significant effect on lane weaving as compared to placebo.

An SDLP of 4.4 cm as compared to control is considered equivalent to the driving impairment associated with a blood alcohol content (BAC) of 0.05%, the threshold for drunk driving in many countries.

The absence of an effect of tasimelteon 20 mg on next-day driving is significant, given the expectation that tasimelteon could be useful in the treatment of jet lag disorder (JLD). A tasimelteon JLD clinical program demonstrates significant benefits in individuals experiencing circadian advances of five to eight hours.

Other indications are similarly amenable to similar treatment without inducing or risking post-sleep/post-dosing/next-day residual effects that could impair next-day performance, including performance in operating a motor vehicle or machinery. Such indications include, for example, circadian rhythm disorders and sleep disorders, such as Non-24-Hour Sleep-Wake Disorder, transient insomnia, chronic insomnia, shift- work disorder, delayed sleep phase disorder, etc. Other such disorders will be apparent to one skilled in the art.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. “Optional” or “optionally” means that the subsequently described event or circumstance may or may not occur, and that the description includes instances where the event occurs and instances where it does not.

The corresponding structures, materials, acts, and equivalents of all means or step plus function elements in the claims below are intended to include any structure, material, or act for performing the function in combination with other claimed elements as specifically claimed. The description of the present disclosure is presented for purposes of illustration and description, but is not intended to be exhaustive or limited to the disclosure in the form disclosed. Many modifications and variations will be apparent to those of ordinary skill in the art without departing from the scope and spirit of the disclosure. Any embodiments chosen and described herein appear in order to best explain the principles of the disclosure and the practical application, and to enable others of ordinary skill in the art to understand the disclosure for various embodiments with various modifications as are suited to the particular use contemplated.

What is claimed is:

1. A method of improving sleep, post-sleep performance, or both in an individual comprising:
determining that the individual intends to or may operate a motor vehicle or machinery during a post-sleep period; and
orally administering to the individual 20 mg of tasimelteon once before bedtime,
wherein the post-sleep period includes a period beginning approximately nine hours after administration of tasimelteon.

2. In a method of treating an individual with tasimelteon, the improvement comprising:
determining that the individual intends to or may operate a motor vehicle or machinery during a post-sleep period; and
orally administering to the individual an amount of tasimelteon effective to improve sleep, post-sleep performance, or both,
wherein the post-sleep period includes a period beginning approximately nine hours after administration of tasimelteon,
thereby avoiding impairment of the individual's driving or operation of machinery during the post-sleep period.
